(19) United States
(12) Patent Application Publication
(10) Pub. No.: US 2011/0054297 A1
(43) Pub. Date: Mar. 3, 2011

(54) MEDICAL SYSTEM

(76) Inventors: Clemens Bulitta, Spardorf (DE); Rainer Graumann, Hochstadt (DE); Karl-Heinz Maier, Nurnberg (DE)

(21) Appl. No.: 12/920,640

(22) PCT Filed: Feb. 26, 2009
(86) PCT No.: PCT/EP09/52309

§ 371 Date: Nov. 22, 2010

(30) Foreign Application Priority Data
Mar. 3, 2008 (DE) 10 2008 012 342.0

(51) Int. Cl. A61B 5/05; A61G 13/00

(52) U.S. Cl. 600/407; 5/601

(57) ABSTRACT

A medical system has an electromagnetic navigation system and a patient bed that supports a patient during a medical procedure assisted by the navigation system. The navigation system includes at least one field coil that is integrated in the patient bed at a defined position.

### BACKGROUND OF THE INVENTION

1. Field of the Invention

The invention concerns a medical system with an electromagnetic navigation system.

2. Description of the Prior Art

Modern medical systems are navigation-assisted in part. Modern optical and electromagnetic navigation systems are available today for such systems and for navigation-assisted medical procedures on a patient. The electromagnetic systems normally have three components. One component is a field generator that serves for the generation of a known electromagnetic field. A second component is sensor coils that serve for the actual position determination of a subject—for example the patient and an instrument—which carries (supports) the sensor coil. In the sensor coils a magnetic field is induced by the field generator, from which magnetic field the position of the sensor coil is then calculated in relation to the field generator. The third component is a monitoring system that monitors the complete processes in the navigation system and accordingly conducts spatial calculations and implements a result output to a suitable interface.

An electromagnetic navigation system in a medical environment is normally used together with an imaging system. The optimal positioning of the field generator hereby represents a problem that has not previously been solved in the best possible manner. Namely, the range of the field generator or its field coil that is usable for the navigation system covers approximately a spatial cube with edge length of only approximately 50 cm. In particular, the optimal ratio of magnetic field strength to positional accuracy is of the greatest importance. The positional accuracy decisively depends on the position of the field generator in the medical environment, for example relative to the imaging system, to the patient and to the instruments that are used.

Today field generators are known as “stand-alone systems” that are positioned in proximity to the region to be observed (thus the “volume of interest”) in a medical procedure (thus a diagnosis, biopsy or operation, for example). For example, the field generators are attached below, to the side or above a patient bed, normally on an adjustable arm. However, accessibility to the patient is most often hindered by this positioning, and therefore the acceptance of electromagnetic navigation systems is reduced.

Since the navigation system is normally clinically applied in a sterile area in the patient treatment—for example a puncture into a suspected structure for biopsy and microscopic diagnostics—the sterility of the navigation system, in particular the field generator, must be ensured in addition to the accessibility of the operating or intervention field. Today complex sterile coverings which often must be customized for to the respective field generator hardware components are required for this purpose. This leads to a further limitation in the flexibility in the use of the navigation system as well as to time delays due to the required sterile covering.

### SUMMARY OF THE INVENTION

An object of the present invention is to provide an improved medical system with electromagnetic navigation system.

The object is achieved by a medical system that includes an electromagnetic navigation system and a patient bed to accommodate a patient during a medical procedure, wherein the medical procedure is assisted by the navigation system. According to the invention, at least one field coil of the navigation system is integrated into the patient bed at a defined spatial position. As used herein, a defined spatial position is a position for which the actual current location at the patient bed is known to the navigation system at all times. Due to the aforementioned short range of the field generator or its field coil, the integration into the patient bed causes the field coil to be positioned very close to the patient without occupying additional, interfering space in the environment of the patient. The access to the patient is improved. An improved work process or and workflow results in the medical procedure, and a shorter intervention time thereby results for the patient. Since the field coil of the navigation system is integrated into the patient bed, the field coil is provided in a sterile manner without having to make use of special coverings for this. The contamination risk for the patient, with connected medical complications (for example wound infections), is reduced by the improved sterility. Since the field coil is already integrated into the patient bed, a shorter preparation time also results for the medical procedure since the field generator or field coil does not first have to be correspondingly placed.

In order to design the medical system in the sense of an optimized workflow during the medical procedure, two alternatives are possible for the arrangement of the field coil at the patient bed.

In the first alternative, the field coil is integrated so as to be stationary in the patient bed, meaning that the field coil is already arranged at a predetermined or known point of the bed in the manufacturing of the patient bed and continuously remains at this location, for example. This location is thus also known to the navigation system as a defined spatial position relative to the bed, and therefore to a patient resting on said bed.

In a second alternative embodiment of the medical system, the field coil is arranged so that it can be displaced below a table plane of the patient bed. The displacement normally ensues parallel to the table plane. The field coil therefore forms a displaceable unit below the table. The displacement capability can be realized via a rail system, for example. This variant offers the advantage that the field coil can be adapted to an optimal position below the patient depending on the current conditions, such as size and bearing of the patient or other medical apparatuses used in the medical system. Since the position of the field coil must also be a defined spatial position for the navigation system for this alternative, the navigation system must be informed about the actual, current location of the field coil at all times. Alternatives also exist for this purpose.

A first alternative for this purpose is an embodiment of a medical system that includes a position detection system to determine the position of the field coil relative to the patient bed. Such a position detection system can include, for example, path sensors that detect the current displacement position of the field coil (for example directions x and y) parallel to the table plane, thus the plane of the bed surface.

An alternative embodiment for the spatial determination of the field coil at the patient bed is a receiver coil of the navigation system that is arranged so as to be stationary at the patient table. Since a relative position between the receiver coil and the field coil can always be determined by the navigation system, the position of the field coil can be determined by the navigation system itself if the absolute position of the receiver coil at the patient table is known. This thus represents a reference position.

For the case of a field coil that can be displaced relative to the patient bed, in a further embodiment of the invention the medical system includes a positioning unit. This serves to detect the spatial position of other components of the medical system that interact with the patient bed during the procedure. For example, this can be one or more imaging systems, therapy units or the like. The positioning unit furthermore serves to determine a suitable position of the field coil relative to the patient bed that takes into account the determined spatial positions of the other components. All additional equipment that is used in the medical system is thus accounted for, and a corresponding, suitable position of the field coil is found at which this is affected as little as possible by the iron mass of the components or interferes as little as possible with a corresponding imaging.

The position of the field coil can be varied or shifted manually or automatically, hand-operated, electrically or pneumatically. In an advantageous embodiment, the medical system therefore has a displacement unit to vary the position of the field coil relative to the patient bed. Such a displacement can also ensue automatically via motors corresponding to a detected displacement of the OP area, for example, such that the field generator or field coil is always automatically located in the optimal position. Such a detection can, for example, ensue using the position of sensor coils that are attached to the operating area (thus to the patient).

Particularly in the case of field coils that are integrated in a fixed manner into the patient bed, the medical system can contain multiple field coils or distributed in the patient bed such that the fields generated by these together cover the entire spatial area associated with the patient bed, which spatial area is occupied by a patient borne on the bed. The field coils can thus cover the entire bed area for all conceivable patients and therefore also do not have to be displaced. The patient on the patient bed also does not have to be shifted toward a field coil.

The medical system can include an imaging system interacting with the patient bed. In a further advantageous embodiment of the invention, the field coils in the patient bed are then arranged at a location that is located outside of that region of the patient bed which can be imaged via the imaging system. For example, this can be the corners of the bed or the location of the column supporting the patient bed. An improved combination of navigated procedures on the patient with intraoperative imaging hereby results.

In a further advantageous embodiment of the invention with the use of the aforementioned imaging system one or more additional field coils of the navigation system can be arranged at the imaging system, so as to be stationary or at a known position relative to the imaging system. The cited field coils communicate wirelessly or via cables with the navigation system and therefore do not have to exhibit a known position relative to the images generated by the imaging system. The position of the imaging system—and therefore of the images generated thereby—is then also known in the coordinate system of the patient bed.

If parts of the patient bed are ferromagnetic, these affect the fields generated by the field coil and the precision of the navigation system. However, such metallic components are known for a given bed and can be included in the algorithm used by navigation system (for example via FEM simulation) or be correspondingly taken into account in table form via a one-time calibration measurement after manufacturing of the bed. However, in an advantageous embodiment of the invention the patient bed is produced from non-ferromagnetic material at least in a portion of the bed that is located in the active field. The active field is the spatial region of the field of the field coil that is usable for the navigation system in which sufficient field strengths are thus provided for a position detection. At least in this region, the patient bed then does not affect the fields generated by the field coil (and therefore the spatial accuracy of the navigation system).

The field-generating portion of a navigation system normally includes only the field coil, but also a correspondingly associated monitoring or control unit. According to a development of the invention, this control unit can also be arranged stationary at the patient bed, for example again in proximity to the OP column or the coupling unit.

The aforementioned (also optional) components such as field coil, monitoring and control unit, position detection system, can—if they are present—already be associated into a bed as integral components of this (i.e. already be integrated in its manufacture). In an advantageous embodiment of the invention, however, at least the field coil—and/or the aforementioned auxiliary components—can also be designed as an attachment kit for different embodiments of patient beds. In other words, a modular navigation assembly set for variable coupling of components (such as the field coil) with differently designed or dimensioned patient beds (from different manufacturers, for example). However, the attachment kit is designed so that the respective position of the field coil relative to the patient bed is again defined in the aforementioned sense (thus is known to the navigation system) for every possible patient bed for which the attachment kit is provided. A correspondingly defined spatial position can also be determined once via a corresponding calibration method, for example after attachment of the attachment kit to the patient bed.

### BRIEF DESCRIPTION OF THE DRAWINGS

FIG. 1 illustrates a medical system having a patient bed and an electromagnetic navigation system in an embodiment having stationary field coils that are integrated into the patient bed.

FIG. 2 illustrates a further embodiment of a medical system with a patient bed and an electromagnetic navigation system having a field generator (field coil) integrated into the patient bed in a manner allowing the field generator to be displaced.

### DESCRIPTION OF THE PREFERRED EMBODIMENTS

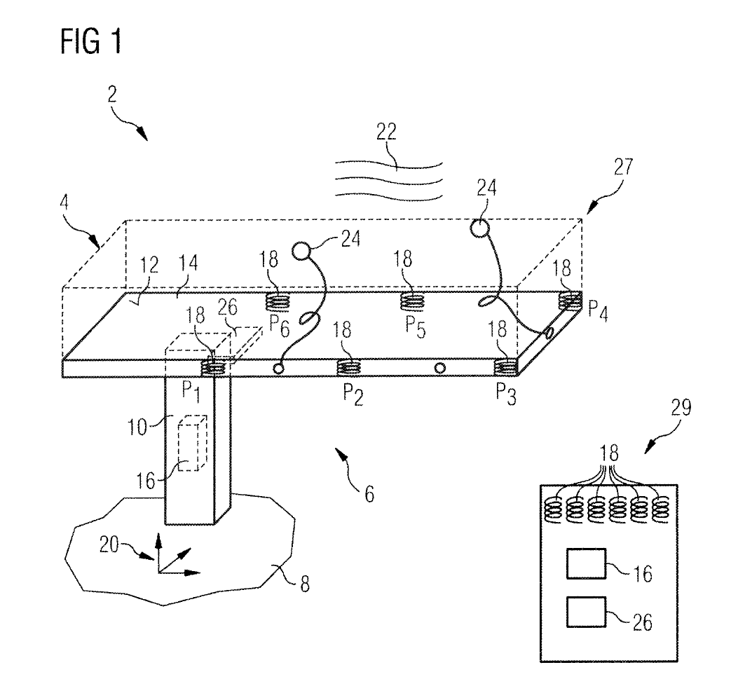

FIG. 1 shows a medical system 2 with a patient bed 4 and an electromagnetic navigation system 6. The patient bed 4 has a pedestal 10 firmly mounted on a floor 8 of a treatment room, on which pedestal 10 a bed surface 12 is mounted in turn whose top side 14 serves to accommodate a patient (not shown).

The navigation system 6 has a field generator 16 that is integrated into the pedestal 10, as well as multiple field coils 18 fed by the field generator 16. The field coils 18 are non-transiently integrated into the bed surface 12, meaning that their spatial positions P1 through P6 are known both relative to the bed surface 12 and relative to an N-coordinate system 20 of the navigation system 6. The N-coordinate system 20 is arranged spatially fixed in the treatment space, thus relative to the floor 8.

The field coils 18 serve to generate an electromagnetic field 22 in the environment of the patient bed 4 in the operation of the navigation system 6. The navigation system 6 moreover has sensor coils 24 that can be connected with the navigation system 6 via connectors 26 that are likewise firmly integrated into the bed surface 12. For example, the sensor coils 24 are attached to the patient (not shown) or an operating tool (not shown).

Due to the presence of the sensor coils 24 in the field 22, their spatial position can be determined. This occurs via a control and monitoring unit 26 belonging to the navigation system 6, which control and monitoring unit 26 is likewise arranged fixed on the patient bed 4. The control and monitoring unit 26 serves for the control or, respectively, readout of field generator 16, field coils 18 and sensor coils 24. The mode of operation of such an electromagnetic navigation system 6 is known and should not be explained in detail here.

Through the arrangement of the field coils at the spatial positions P1 through P6, the field 22 in FIG. 1 covers at least the entire spatial region 27 above the patient bed 4. This spatial region 27 is that region which can be taken up by an arbitrary patient (not shown) that is placed on the patient bed 4. The field thus covers the entire region that is of interest for a navigated procedure on the patient.

In an alternative embodiment, the field coils 18, the field generator 16 and the control and monitoring unit 26 are shown as part of an attachment kit 29. The attachment kit 29 is distributed separately from the patient bed 4 by the manufacturer of the navigation system 6; however, it is tailored to the specific patient bed 4. Given installation of the medical system 2, the parts of the attachment kit 29 are attached to the patient bed 4 in order to upgrade this into a corresponding system (shown in FIG. 1) with integrated navigation system 6.

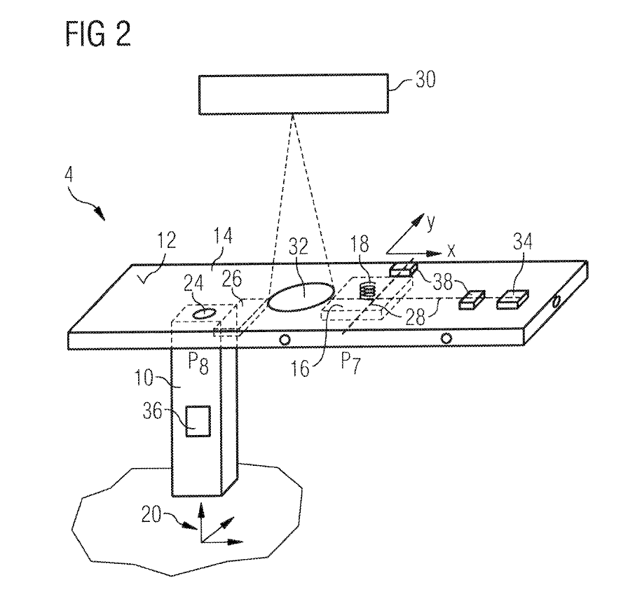

FIG. 2 shows an alternative embodiment of a patient bed 4 in which a single field generator 16 is provided with an integrated field coil 18 instead of the multiple field coils 18 from FIG. 1. The field generator 16 is arranged on a rail system 26 that can move below the bed surface 12. Due to the rail system 28, the field generator 16 and the field coil 18 can thus be displaced in the plane of the directions x and y, thus parallel to the top side 14 of the bed surface 12.

An imaging system 30 which is aligned on an imaging region 32 of the patient bed 4—i.e. can expose a patient (not shown) on the patient bed 4 in the imaging region 32—is additionally shown in FIG. 2. Since both the control and monitoring unit 26 and the field generator 16 would interfere with the imaging of the imaging system 30, these are arranged at the patient bed 4 such that they do not lie in the imaging region 32.

Since the N-coordinate system 20 should again be stationary (for example should be anchored to the floor 8) but the field generator 16 can be displaced, the current position P₇ of the field generator 16—and therefore the field coil 18—is determined by a position detection system 34. In a first embodiment, this determines the coordinates of the field coil 18 relative to the directions x and y using mechanical sensors (not shown) on the rail system 28.

In an alternative embodiment, a sensor coil 24 is mounted fixed at a known position P₈ at the patient bed 3. The position detection of the field coil 18 then occurs in the control and monitoring unit 26 in that the position of the sensor coil 24 relative to the generator 16 or the field coil 18 is determined, and the current coordinates of the directions x and y at which the field coil 18 is currently located are determined from this.

Moreover, a positioning unit 36 is integrated in FIG. 2, which positioning unit 36 detects the position of the patient bed 4, of the imaging system 30 and of other components (not shown) of the medical system 2 and from this determines an optimal position for the field generator 16 at which it does not interfere with the imaging and the field coil 18 generates an optimal field 22 in order to cover the spatial region of interest for additional sensor coils (not shown), for example at the patient. With the use of the rail system 28, the field coil 18 is automatically moved to the correspondingly determined position by an automatic adjustment unit 38.

Although modifications and changes may be suggested by those skilled in the art, it is the intention of the inventors to embody within the patent warranted heron all changes and modifications as reasonably and properly come within the scope of their contribution to the art.

We claim as our invention:

1.-11. (canceled)

12. A medical system comprising:
a patient bed adapted to accommodate a patient thereon during a medical procedure, said patient table defining a table plane beneath the patient;
a navigation system configured to provide position information to assist in implementing said medical procedure, said navigation system comprising at least one field coil; and
said at least one field coil being integrated into the patent bed at a defined spatial position known to the navigation system, said field coil being mounted at said patient bed to allow displacement of said field coil beneath said table plane of said patient bed.

13. A medical system as claimed in claim 12 comprising a field generator that emits a signal to said field coil that causes said field coil to radiate an electromagnetic field, and wherein said field generator is displaceable together with said field coil beneath said table plane of the patient bed.

14. A medical system as claimed in claim 12 wherein said navigation system comprises a computerized position detection system that determines the position of the field coil relative to the patient bed.

15. A medical system as claimed in claim 14 wherein said navigation system comprises a receiver coil that detects an electromagnetic field radiated by said field coil and supplies a signal corresponding to the detected electromagnetic field to said position detection system, said receiver coil being mounted stationary at said patient bed.

16. A medical system as claimed in claim 12 comprising a positioning unit configured to detect respective spatial positions of components that interact with said patient bed during said medical procedure, said positioning unit being configured to determine a position of the field coil relative to the patient bed dependent on the respective spatial positions, and being configured to decouple said navigation system from said components.

17. A medical system as claimed in claim 12 comprising an adjustment unit mechanically connected to said field coil that adjusts a position of said field coil relative to the patient bed.

18. A medical system as claimed in claim 12 comprising a plurality of field coils integrated in said patient bed, each of said field coils radiating an electromagnetic field, with the respective radiated electromagnetic fields, covering an entirety of a spatial region associated with the patient bed, said spatial region being occupied by the patient on the patient bed.

19. A medical system as claimed in claim 12 comprising an imaging system configured to generate an image of an imaging region encompassing the patient on the patient bed, and said field coil being located at said patient bed outside of said imaging region.

20. A medical system as claimed in claim 19 wherein said navigation system comprises an additional field coil located at said imaging system.

21. A medical system as claimed in claim 12 wherein said field coil has an active range associated therewith, and wherein said patient bed is comprised of non-ferromagnetic material at least in portion of said active range.

22. A medical system as claimed in claim 12 comprising a control unit that is located stationary at said patient bed.

23. A medical system as claimed in claim 12 wherein said field coil is integrated at said patient bed by in situ attachment to said patient bed.
